(19) United States
(12) Patent Application Publication
(10) Pub. No.: US 2022/0378963 A1
(43) Pub. Date: Dec. 1, 2022

(54) HAND-HELD OPERATOPM CLEANING, STERIIZATION AND DISINFECTION ATOMIZING DIFFUSER

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 17/330,314

(22) Filed: May 25, 2021

(51) Int. Cl. A61L 2/22; B05B 7/06; B05B 11/00; A61L 2/26

(52) U.S. Cl. CPC A61L 2/22; B05B 7/066; B05B 11/3042; A61L 2/26; A61L 2202/16; A61L 2202/15; A61L 2202/14; A61L 2202/121; A61L 2202/11; A61L 2202/17

(57) ABSTRACT

A hand-held operation cleaning, sterilization and disinfection atomizing diffuser, provided that the user conveniently holds a handle of the diffuser to operate a control unit to provide power to an air extraction device and a pumping control device to pump the cleaning, sterilizing, disinfecting or/and deodorizing liquid out of a liquid storage tank to expel via a water jet, and the gas extracted from the air extraction forms a jet stream through an air jet, and the liquid sprayed by the water jet is broken by the jet stream of the air jet at a high speed into fine droplets and atomized to clean, sterilize, disinfect or deodorize towards a target.

### BACKGROUND OF THE INVENTION

### 1. Field of the Invention

The present invention relates to cleaning, sterilization and disinfection atomizing diffusers and more specifically, to a hand-held operation cleaning, sterilization and disinfection atomizing diffuser that can be operated by one hand.

### 2. Description of the Related Art

Existing environment, personnel cleaning, deodorization, sterilization, disinfection sprayers usually use bottled sprayers, oscillatory disc atomization disinfection machines, or knapsack sprayers. The spray effect of the bottled sprayer device is limited and cannot be reused. The distance of the atomized disinfectant sprayed by the oscillatory disc atomization disinfection machine is limited, and it is difficult to spray the target object. In addition, the power cords of these electrical appliances are connected to electrical sockets with restrictions on their use, and the knapsack sprayer makes it inconvenient for users to use.

In addition, cleaning and disinfection use bleach, alcohol disinfectant and other chemicals, which cause irritation, corrosion, residue, or allergies or discomfort to the human body. The use of alcohol spraying device often makes the alcohol droplets remain on the table. The safety issue should not be careless. These liquid alcohol or bleach are not suitable for spraying in the space. Therefore, the scope of cleaning is difficult to take into account the three-dimensional space or the indoor furnishings above the ground.

### SUMMARY OF THE INVENTION

The present invention has been accomplished under the circumstances in view. It is therefore an object of the present invention to provide a hand-held operation cleaning, sterilization and disinfection atomizing diffuser that can be operated by one hand.

The hand-held operation cleaning, sterilization and disinfection atomizing diffuser comprises a housing, a wind collecting hood device, an air extraction device, a liquid storage tank, a pumping control device, a first air pipe, a first liquid delivery pipe, a second liquid delivery pipe, a power supply unit and a control unit.

The housing comprises an opening formed on one side thereof, and a first hole formed on an opposite side thereof.

The wind collecting hood device comprises a second hole, a third hole, a second air pipe and a nozzle. The second hole is formed on an upper side of the wind collecting hood device. The third hole and the second air pipe are respectively formed on one side of the wind collecting hood device. The nozzle is combined in the second hole. The nozzle comprises a plurality of water jets and a plurality of air jets. The water jets and the air jets are respectively distributed in the nozzle. The air jets are connected to the inside of the wind collecting hood device. The air extraction device is combined inside the wind collecting hood device. The wind collecting hood device is combined inside the housing. The second hole of the wind collecting hood device has one side thereof combined in the first hole.

The liquid storage tank comprises an air inlet and a water outlet. The air inlet and the water outlet are respectively formed on an upper side of the liquid storage tank.

The liquid storage tank, the pumping control device, the first air pipe, the first liquid delivery pipe, the second liquid delivery pipe, the power supply unit and the control unit are combined inside the housing. The liquid storage tank is located under the wind collecting hood device. The first air pipe has one end thereof connected to the second air pipe, and an opposite end thereof connected to the air inlet. The first liquid delivery pipe has one end thereof connected to the water outlet, and an opposite end thereof connected to one side of the pumping control device. The second liquid delivery pipe has one end thereof inserted through the third hole and connected to an opposite side of the pumping control device, and an opposite end thereof connected to the water jet.

The control unit comprises a control circuit coupled with the power supply unit. The control unit is electrically connected to the air extraction device and the pumping control device.

In application, the user can hold the atomizing diffuser, and the power supply unit can store power, without the need for an external power cord to fetter the atomizing diffuser. Operate the control unit to supply the power of the power supply unit to the air extraction device and the pumping control device. The air extraction device sucks gas into the wind collecting hood device through the opening, and the gas accumulates in the wind collecting hood device to reach the liquid storage tank through the second air pipe, the first air pipe and the air inlet. The space inside the liquid storage tank is filled with some liquid, such as cleaning, sterilizing, disinfecting or deodorizing liquid. The gas forms a pushing pressure on the liquid in the space in the liquid storage tank, and the pumping control device is easier to pump the liquid in the liquid storage tank. The gas forms a pushing pressure on the liquid in the space of the liquid storage tank, which helps the cleaning, sterilizing, disinfecting or deodorizing liquid to quickly and continuously pass through the water outlet, the first liquid delivery pipe, and be transported through the pumping control device and the second liquid delivery pipe and then sprayed through the water jets.

On the other hand, the accumulated air pressure in the wind collecting hood device quickly and continuously forms a jet stream through the air jets, and quickly ejects gas. The cleaning, sterilizing, disinfecting or deodorizing liquid sprayed by the fast sprayed gas and the water jets merges and the gas breaks the liquid at a high speed and disperses into fine water droplets. At the same time, the high-speed gas constantly impacts and rubs the liquid, and the liquid is continuously dispersed into fine water molecules in an instant, and atomization is achieved. The atomizing diffuser can form a long-distance spray to spray the spraying target, as the three-dimensional space, the ground, the indoor furnishings, especially the indoor furnishings, to sterilize, clean, deodorize, and eliminate viruses.

In one embodiment of the hand-held operation cleaning, sterilization and disinfection atomizing diffuser of the present invention, the liquid storage tank is filled with electrolyzed alkaline ionized water, and the alkaline ionized water spray produced is non-toxic, non-irritating, and non-residual. It uses gaseous spray to clean, sterilize, deodorize, and disinfect the environment to purify the environmental space, which is safe for the environment and does not harm human health.

### BRIEF DESCRIPTION OF THE DRAWINGS

FIG. 1 is an oblique top elevational view of a hand-held operation cleaning, sterilization and disinfection atomizing diffuser in accordance with the present invention.

FIG. 2 corresponds to FIG. 1 when viewed from another angle.

FIG. 3 is an exploded view of the hand-held operation cleaning, sterilization and disinfection atomizing diffuser in accordance with the present invention.

FIG. 4 is a schematic sectional view of the hand-held operation cleaning, sterilization and disinfection atomizing diffuser in accordance with the present invention.

FIG. 5 is an internal schematic diagram of the hand-held operation cleaning, sterilization and disinfection atomizing diffuser in accordance with the present invention.

### DETAILED DESCRIPTION OF THE PREFERRED EMBODIMENT

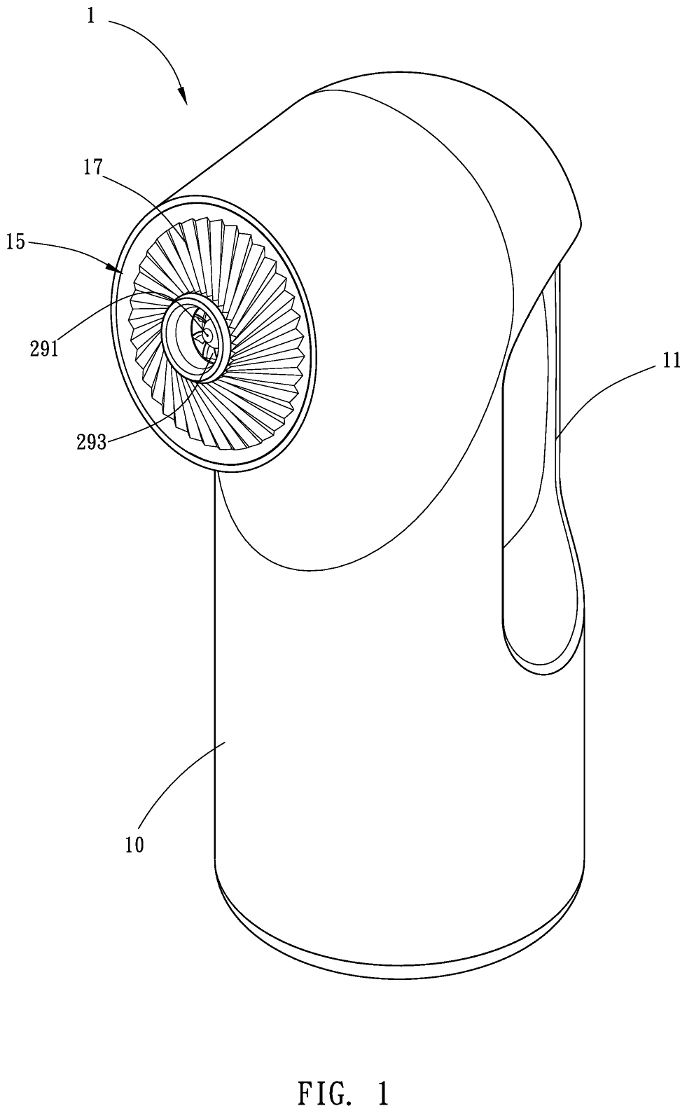

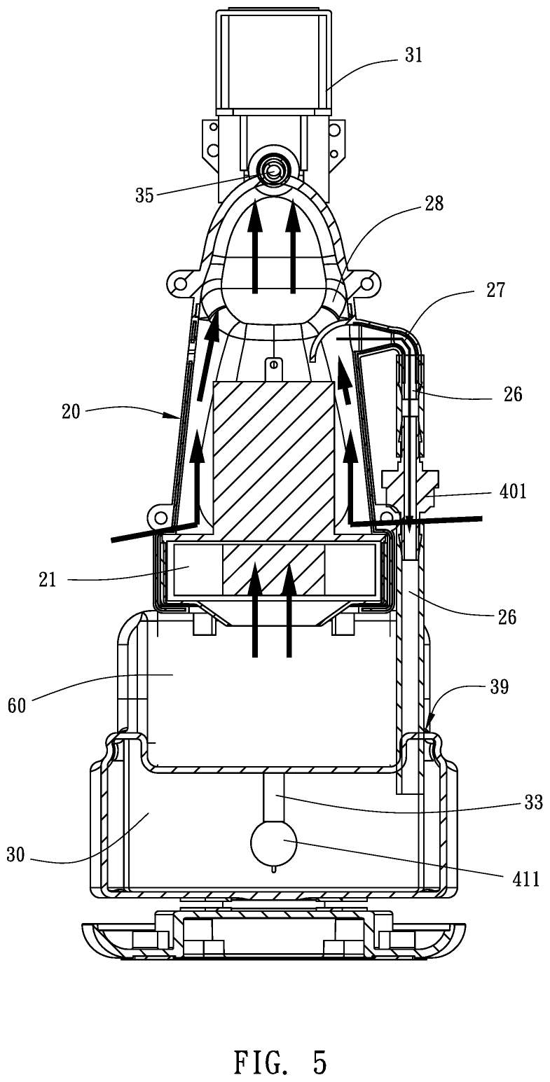

Referring to FIGS. 1-5, a hand-held operation cleaning, sterilization and disinfection atomizing diffuser in accordance with the present invention is shown. The hand-held operation cleaning, sterilization and disinfection atomizing diffuser 1 comprises a housing 10, a wind collecting hood device 20, an air extraction device 21, a liquid storage tank 30, a pumping control device 31, a first air pipe 26, a first liquid delivery pipe 33, a second liquid delivery pipe 35, a power supply unit 60, and a control unit 70.

The housing 10 comprises an opening 13 formed on one side thereof, and a first hole 15 formed on an opposite side thereof.

The wind collecting hood device 20 comprises a second hole 23, a third hole 25, a second air pipe 27, and a nozzle 29. The second hole 23 is formed on the top side of the wind collecting hood device 20. The third hole 25 and the second air pipe 27 are respectively formed on one side of the wind collecting hood device 20. The nozzle 29 is combined in the second hole 23. The nozzle 29 comprises a plurality of water jets 291 and a plurality of air jets 293. The water jets 291 and the air jets 293 are respectively distributed in the nozzle 29. The air jets 293 communicate with the inside of the wind collecting hood device 20. The air extraction device 21 is combined in the wind collecting hood device 20. The wind collecting hood device 20 is combined in the housing 10. The second hole 23 of the wind collecting hood device 20 is integrated in the first hole 15.

The liquid storage tank 30 comprises an air inlet 37, a water outlet 39. The air inlet 37 and the water outlet 39 are respectively formed on the upper side of the liquid storage tank 30.

The liquid storage tank 30, the pumping control device 31, the first air pipe 26, the first liquid delivery pipe 33, the second liquid delivery pipe 35, the power supply unit 60 and the control unit 70 are combined in the housing 10. The liquid storage tank 30 is located under the wind collecting hood device 20. The first air pipe 26 has one end thereof connected to the second air pipe 27, and an opposite end thereof connected to the air inlet 37. The first liquid delivery pipe 33 has one end thereof connected to the inner end of the water outlet 39, and an opposite end thereof connected to one side of the pumping control device 31. The second liquid delivery pipe 35 has one end thereof inserted through the third hole 25 and connected to the other side of the pumping control device 31, and an opposite end thereof connected to the water jets 291.

The control unit 70 comprises a control circuit. The control unit 70 is electrically connected to the air extraction device 21 and the pumping control device 31. The power supply unit 60 is coupled to the control unit 70.

Thereby, the user can hold the atomizing diffuser 1, and operate the control unit 70 to provide the power supply of the power supply unit 60 to the air extraction device 21 and the pumping control device 31. The pumping control device 31 pumps the cleaning, sterilizing, disinfecting or deodorizing liquid out of the liquid storage tank 30. The cleaning, sterilizing, disinfecting or deodorizing liquid quickly and continuously passes through the water outlet 39, the first liquid delivery pipe 33, is transported through the pumping control device 31, and is ejected through the water jets 291 via the second liquid delivery pipe 35.

On the other hand, the accumulated air pressure inside the wind collecting hood device 20 rapidly and continuously forms a jet flow through the air jets 293, and rapidly ejects gas. The rapidly sprayed gas is combined with the cleaning, sterilizing, disinfecting or deodorizing liquid sprayed by the water jets 291. The gas smashes the liquid at high speed and breaks it into fine water droplets. At the same time, the high-speed gas constantly impacts and rubs the liquid, and the liquid is continuously dispersed into fine water molecules in an instant, and the cleaning, sterilizing, disinfecting or deodorizing liquid is atomized to clean, sterilize, disinfect or deodorize the spray target.

In one embodiment of the present invention, the user can hold the atomizing diffuser 1, and the power supply unit can store power, without the need for an external power cord to fetter the atomizing diffuser 1. Operate the control unit 70 to supply the power of the power supply unit 60 to the air extraction device 21 and the pumping control device 31. The air extraction device 21 sucks gas into the wind collecting hood device 20 through the opening 13, and the gas accumulates in the wind collecting hood device 20 to reach the liquid storage tank 30 through the second air pipe 27, the first air pipe 26 and the air inlet 37. The space inside the liquid storage tank 30 is filled with some liquid, such as cleaning, sterilizing, disinfecting or deodorizing liquid. The gas forms a pushing pressure on the liquid in the space in the liquid storage tank 30, and the pumping control device 31 is easier to pump the liquid in the liquid storage tank 30. The gas forms a pushing pressure on the liquid in the space of the liquid storage tank 30, which helps the cleaning, sterilizing, disinfecting or deodorizing liquid to quickly and continuously pass through the water outlet 39, the first liquid delivery pipe 33, and be transported through the pumping control device 31 and the second liquid delivery pipe 35 and then sprayed through the water jets 291.

On the other hand, the accumulated air pressure in the wind collecting hood device 30 quickly and continuously forms a jet stream through the air jets 293, and quickly ejects gas. The cleaning, sterilizing, disinfecting or deodorizing liquid sprayed by the fast sprayed gas and the water jets 291 merges and the gas breaks the liquid at a high speed and disperses into fine water droplets. At the same time, the high-speed gas constantly impacts and rubs the liquid, and the liquid is continuously dispersed into fine water molecules in an instant, and atomization is achieved. The atomizing diffuser 1 can form a long-distance spray to spray the spraying target, as the three-dimensional space, the ground, the indoor furnishings, especially the indoor furnishings, to sterilize, clean, deodorize, and eliminate viruses.

The cleaning, sterilizing, disinfecting or deodorizing liquid can be, for example, alkaline ionized water or various environmental cleaning and purification preparations, etc., or adding various natural sterilizing and deodorizing agents to the liquid for atomization. In the alkaline ionized water embodiment, the automizing diffuser 1 uses pure water electrolyzed hydroxide ionized water, for example, pH value of about 10 to 12.5, preferably, a pH value of 12.5. The high-alkaline hydroxide ionized water molecules are sprayed and output to the external environmental space, which is used for sterilization, cleaning, deodorization, virus elimination or air purification of indoor furnishings. The safety of use does not affect human health.

In some embodiments, the air extraction device 21 is, for example, a suction fan motor used in a vacuum cleaner or various high-speed performance fan motors. The suction fan motor can instantly reach high speed and high suction volume and deliver it to the air jets 293 to produce a high-speed jet to break up the liquid, or the high-speed jet can be delivered to the inside of the liquid storage tank 30. In the embodiment, the first air pipe 26, the first liquid delivery pipe 33 or the second liquid delivery pipe 35 can be made of flexible pipes for convenient transition and connection, such as silicone, plastic, rubber, etc.

Please refer to FIGS. 1-4. In one embodiment of the present invention, the atomizing diffuser 1 further comprises a handle 11 formed on one side of the housing 10, for example, the handle 11 is formed on one side of the housing 10 opposite to the first hole 15.

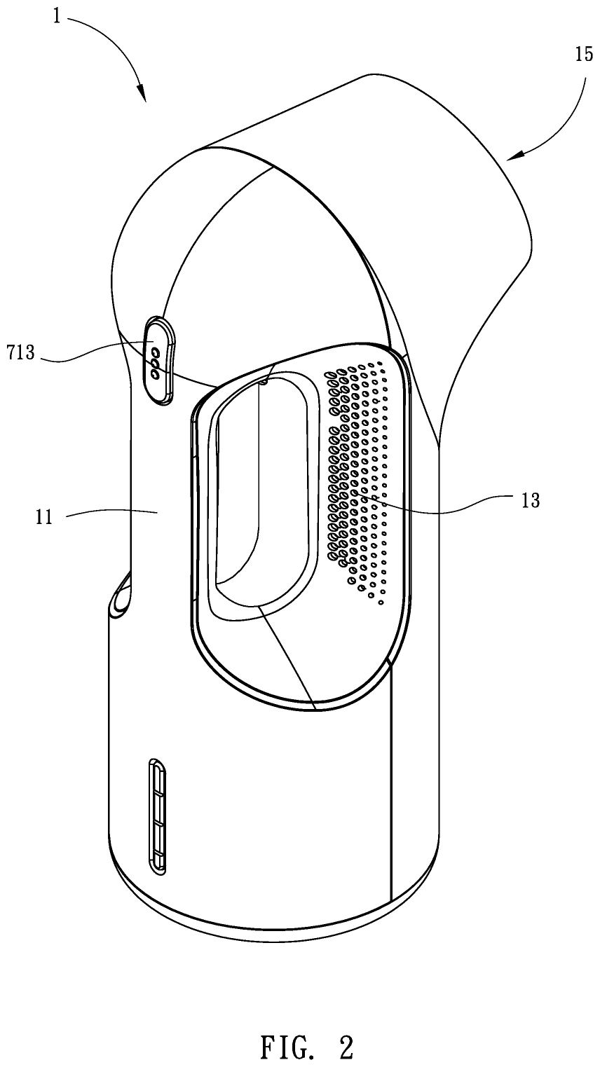

Please refer to FIGS. 2-4. In one embodiment of the present invention, the atomizing diffuser 1 further comprises an electric switch control device 71. The electric switch control device 71 comprises a circuit device 711 of a power switch PCB and a combined switch button 713. The control unit 70 is coupled to the electric switch control device 71. The electric switch control device 71 is combined in one side of the housing 10. The switch button 713 is adjacent to one side of the handle 11. By this, while the user's hand is holding the handle 11, the user's finger can easily touch the switch button 713, so as to control the opening or closing signal command of the atomizing diffuser 1 of the present invention to the control unit 70 for starting or turning off the air extraction device 21 or the pumping control device 31.

Please refer to FIGS. 2-4. In one embodiment of the present invention, the pumping control device 31 further comprises a control valve 311 and a pump 313. The control valve 311 can be, for example, the switch of a solenoid valve or a solenoid valve of a water dispenser. The control valve 311 is combined with the pump 313 to operate the pump on or off, so as to control the liquid transportation inside the liquid storage tank 30 through the first liquid delivery pipe 33, the pump 313, the second liquid delivery pipe 35 and the water jets 291.

Please refer to FIGS. 1, 3 and 4. In one embodiment of the present invention, the water jets 291 are formed in the middle of the nozzle 29, and the air jets 293 are formed on the outside of the water jets 291. In the drawings, the air jets 293 are arranged around the water jets 291. In this way, the liquid sprayed by the water jets 291 is merged by rapidly spraying gas around it. The liquid can be, for example, cleaning, sterilizing, disinfecting or deodorizing liquid. The air jet smashes the liquid into fine water droplets at high speed. At the same time, the high-speed jet constantly impacts and rubs the liquid, and the liquid continuously disperses into fine water molecules in an instant to achieve cleaning, sterilizing, disinfecting or deodorizing liquid atomization to effectively clean, sterilize, disinfect or deodorize the spray target.

In one embodiment of the present invention, the water jets 291 protrude from the surface of the nozzle 29, and the height of the water jets 291 is higher than the air jets 293, that is, the water jets 291 are located in front of the air jets 293. The liquid ejected by this water jets 291 is in front of or above the air jets 293. The jetted air blows the liquid behind or below the liquid to form a better atomization effect. At the same time, the small atomized water molecules of the liquid are blown forward or upward to transport the liquid atomized water molecules of the above-mentioned cleaning, sterilizing, disinfecting or deodorizing liquid effectively to the target, such as curtains at high places, ceilings, deep gaps, cabinets or pipes, etc. that is not easy to reach by spraying.

Please refer to FIGS. 1, 3 and 4. In one embodiment of the present invention, the direction of the water jets 291 and the air jets 293 of the hand-held operation cleaning, sterilization and disinfection atomizing diffuser are parallel. The water jets 291 are used to spray liquid, and the air jets 293 are used to spray diffuse gas. The spray direction of the water jets 291 and the air jets 293 are the same or parallel. In this way, the air jets and the liquid merge. The air jets can push the liquid forward or upward, and the air jets can frictionally blow the liquid away to form tiny water molecules and the atomization effect is good.

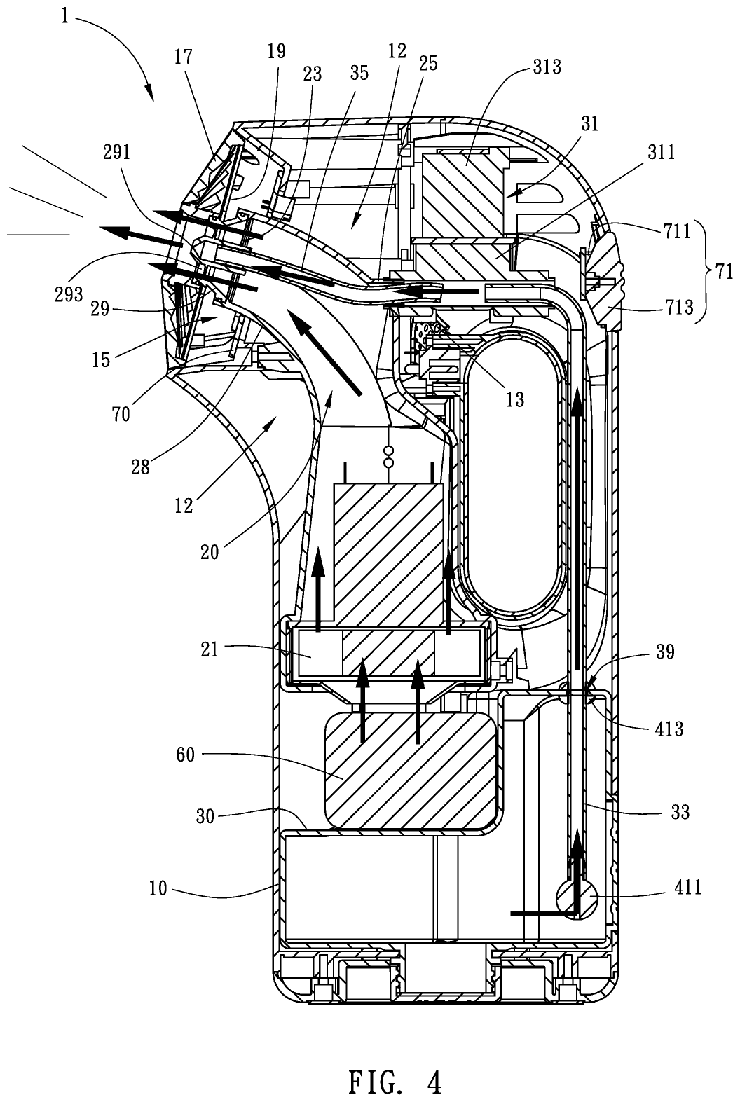

Please refer to FIG. 4. In one embodiment of the present invention, the free end of the outer side of the water jets 291 is a conical water jet structure, and the above-mentioned cleaning, sterilizing, disinfecting or deodorizing liquid passes through the constriction of the conical water jet structure, and the liquid will accelerate the ejection from the water jets 291 to reach the target in the distance.

Please refer to FIGS. 1, 3 and 4. In one embodiment of the present invention, the air jets 293 are formed at intervals on the outside of the water jets 291. The above-mentioned cleaning, sterilizing, disinfecting or deodorizing liquid is sprayed from the water jets 291, and the air jets 293 blow the air jets evenly to the liquid, which can improve the atomization effect.

Please refer to FIGS. 1, 3 and 4. In one embodiment of the present invention, the housing 10 has a portion protruding from one side of the first hole 15 to define a cavity 12, and the front end of the cavity 12 forms the first hole 15. The cavity 12, the first hole 15, the wind collecting hood device 20 and the second hole 23 are configured to protrude outward in one direction, so that when the user holds the handle 11 and operates the atomizing diffuser 1, the atomized spray of the nozzle 29 inside the first hole 15 and the second hole 23 can be sprayed towards the target direction with the cleaning, sterilizing, disinfecting or deodorizing liquid.

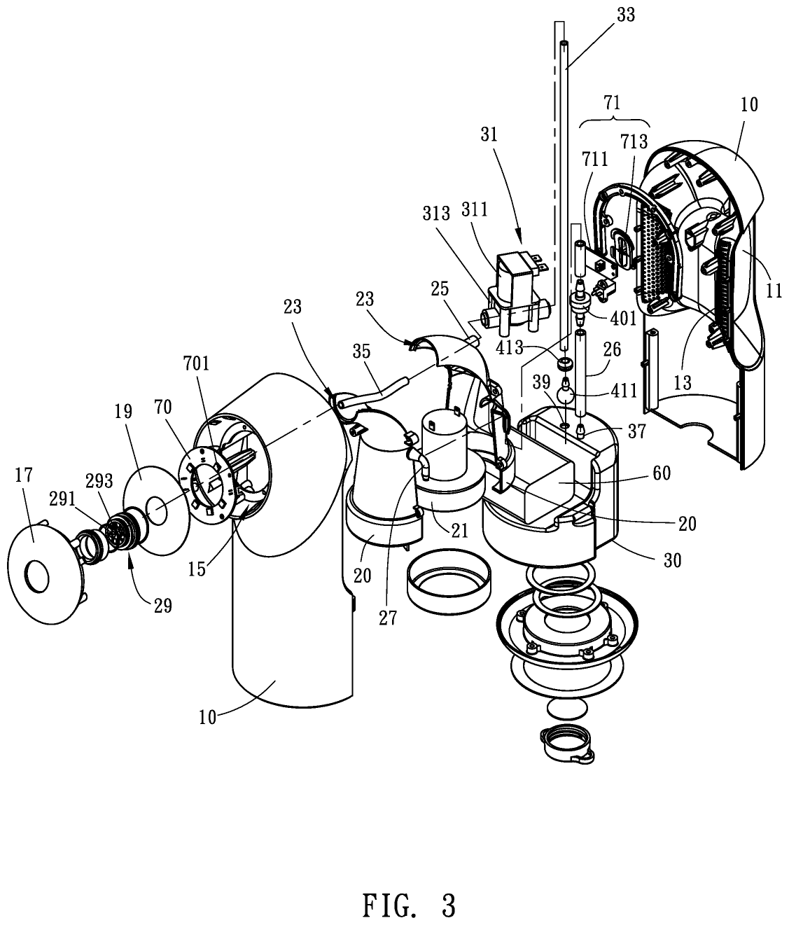

Please refer to FIGS. 3-5. In one embodiment of the present invention, the wind collecting hood device 20 of the hand-held operation cleaning, sterilization and disinfection atomizing diffuser 1 comprises a conical curved tube 28, which is curved and tapered from the lower side to the upper side of the wind collecting hood device 20. The conical curved tube 28 of the wind collecting hood device 20 extends to the smallest tube diameter, the end of the smallest tube diameter forms the second hole 23. The conical curved tube 28 is combined inside the cavity 12, and the second hole 23 is formed at the end of the smallest tube diameter of the tapered wind collecting hood device 20. Therefore, the nozzle 29 can cooperate with the second hole 23 to reduce the volume. The gas conveyed by the air extraction device 21 forms a greater gas pressure to the air jets 293 through the air collection effect of the curved tube, resulting in a larger jet flow to further effectively blow the liquid sprayed by water jets 291 to enhance the atomization effect.

Please refer to FIGS. 1-4. In one embodiment of the present invention, in the hand-held operation cleaning, sterilization and disinfection atomizing diffuser 1, the cavity 12, the first hole 15, the wind collecting hood device 20 and the nozzle 29 in the second hole 23 are pitched upward at an angle relative to the housing 10. The pitch angle is about 1 degree to 89 degrees, such as 30, 35, 40, 45, 50, 55, 60, 65.70 degrees. The liquid or gas of the nozzle 29 will fall down as soon as it is ejected under the influence of gravity. The pitch angle provides that the atomizing diffuser 1 is sprayed in the upward direction, and the atomized liquid molecules can be transported forward for a long distance to the target, and the atomized water molecules will not fall too quickly.

Please refer to FIGS. 3-5. In one embodiment of the present invention, the first air pipe 26 comprises a water stop check valve 401, which is combined on either side of the first air pipe 26. The gas can flow to the liquid storage tank 30 through the water stop check valve 401. he liquid in the liquid storage tank 30 will not flow back into the wind collecting hood device 20 through the water stop check valve 401.

Please refer to FIGS. 3-5. In one embodiment of the present invention, the first liquid delivery pipe 33 comprises a gravity ball 411 with a hole, and a fixing plug 413. The fixing plug 413 is combined in the water outlet 39. One end of the first liquid delivery pipe 33 is connected to the gravity ball 411. The first liquid delivery pipe 33 is inserted through the fixing plug 413 and connected to the inner side of the water outlet 39. The gravity ball 411 also movably maintains one end of the first liquid delivery pipe 33 on the bottom side of the liquid storage tank 30 by gravity. Thereby, when the liquid storage tank 30 is at a low water level, the first liquid delivery pipe 33 can effectively absorb the liquid.

Please refer to FIGS. 1, 3 and 4. In one embodiment of the present invention, the atomizing diffuser 1 further comprises a light-transmissive mid-transparent umbrella-shaped cover 17, and a mid-pass diffuser 19. The control unit 70 comprises a light source 701. The control unit 70, the light-transmissive mid-transparent umbrella-shaped cover 17 and the mid-pass diffuser 19 are combined inside the first hole 15. The mid-pass diffuser 19 is combined with the light source 701 of the control unit. The light-transmissive mid-transparent umbrella-shaped cover 17 is combined with the upper side of the mid-pass diffuser 19. The control unit 70 connects the power of the power supply unit 60 to the light source 701. The light source 701 can be an LED lamp, an incandescent lamp bulb, or a CFL series fluorescent lamp. In this embodiment, an LED lamp is used to illuminate the mid-pass diffuser 19 to form a uniform light effect. The mid-pass diffuser 19 is uniformly diffused on the light-transmissive mid-transparent umbrella-shaped cover 17 to produce a light decorative effect.

In an embodiment of the hand-held operation cleaning, sterilization and disinfection atomizing diffuser of the present invention, the user uses cleaning, sterilizing, disinfecting or deodorizing liquid, for example, selects alkaline ionized water with a pH value of 10.0 or more, for example, a pH value of 10.5, 11, 11.5, 12, 12.5 pure water electrolysis alkaline ionized water. In the preferred embodiment, the pH value of hydroxide ionized water from pure water electrolysis is 10 to 12.5. Alkaline ionized water has a peeling effect, which can neutralize static electricity, float away dirt, alkalizate, and decompose oil or protein. It has stronger permeability than ordinary water, and has a good cleaning and sterilization effect.

Although particular embodiments of the present invention have been described in detail for purposes of illustration, various modifications and enhancements may be made without departing from the spirit and scope of the invention. Accordingly, the invention is not to be limited except as by the appended claims.

What the invention claimed is:

1. A hand-held operation cleaning, sterilization and disinfection atomizing diffuser, comprising a housing, a wind collecting hood device, an air extraction device, a liquid storage tank, a pumping control device, a first air pipe, a first liquid delivery pipe, a second liquid delivery pipe, a power supply unit and a control unit, wherein:
said housing comprises an opening formed on one side thereof and a first hole formed on an opposite side thereof;
said wind collecting hood device comprises a second hole, a third hole, a second air pipe and a nozzle, said second hole being formed on an upper side of said wind collecting hood device, said third hole and said second air pipe being respectively formed on one side of said wind collecting hood device, said nozzle being combined in said second hole, said nozzle comprising a plurality of water jets and a plurality of air jets, said water jets and said air jets being respectively distributed in said nozzle, said air jets being connected to the inside of said wind collecting hood device, said air extraction device being combined inside said wind collecting hood device, said wind collecting hood device being combined inside said housing, said second hole of said wind collecting hood device having one side thereof combined in said first hole;
said liquid storage tank comprises an air inlet and a water outlet, said air inlet and said water outlet being respectively formed on an upper side of said liquid storage tank;
said liquid storage tank, said pumping control device, said first air pipe, said first liquid delivery pipe, said second liquid delivery pipe, said power supply unit and said control unit are combined inside said housing, said liquid storage tank being located under said wind collecting hood device, said first air pipe having one end thereof connected to said second air pipe and an opposite end thereof connected to said air inlet, said first liquid delivery pipe having one end thereof connected to said water outlet and an opposite end thereof connected to one side of said pumping control device, said second liquid delivery pipe having one end thereof inserted through said third hole and connected to an opposite side of said pumping control device and an opposite end thereof connected to said water jet.
said control unit comprises a control circuit coupled with said power supply unit, said control unit being electrically connected to said air extraction device and said pumping control device.

2. The hand-held operation cleaning, sterilization and disinfection atomizing diffuser as claimed in claim 1, further comprising a handle and an electric switch control device, said handle being formed on one side of said housing, said electric switch control device comprising a circuit device and a switch button combined with said circuit device, said electric switch control device being combined on one side of said housing, said switch button being adjacent to one side of said handle, said control unit being coupled with said electric switch control device.

3. The hand-held operation cleaning, sterilization and disinfection atomizing diffuser as claimed in claim 1, wherein said pumping control device further comprises a control valve and a pump, said control valve being combined with said pump, said control valve being used to control the opening and closing of said pump.

4. The hand-held operation cleaning, sterilization and disinfection atomizing diffuser as claimed in claim 1, wherein said water jets are formed in the middle of said nozzle; said air jets are formed on the outside of said water jets.

5. The hand-held operation cleaning, sterilization and disinfection atomizing diffuser as claimed in claim 1, wherein said water jets protrude from a surface of said nozzle, and the height of said water jets is higher than said air jets.

6. The hand-held operation cleaning, sterilization and disinfection atomizing diffuser as claimed in claim 4, wherein said water jets protrude from a surface of said nozzle, and the height of said water jets is higher than said air jets.

7. The hand-held operation cleaning, sterilization and disinfection atomizing diffuser as claimed in claim 5, wherein said air jets are spaced at intervals outside said water jets.

8. The hand-held operation cleaning, sterilization and disinfection atomizing diffuser as claimed in claim 6, wherein said air jets are spaced at intervals outside said water jets.

9. The hand-held operation cleaning, sterilization and disinfection atomizing diffuser as claimed in claim 7, wherein said housing has a portion thereof protruding from one side of said first hole to define a cavity, and said cavity having a from end thereof forming said first hole; said wind collecting hood device comprises a conical curved tube curved and tapered from a lower side to an upper side of said wind collecting hood device, said conical curved tube of said wind collecting hood device extending to the smallest tube diameter and the end of the smallest tube diameter forming said second hole, said conical curved tube being combined inside said cavity.

10. The hand-held operation cleaning, sterilization and disinfection atomizing diffuser as claimed in claim 8, wherein said housing has a portion thereof protruding from one side of said first hole to define a cavity, and said cavity having a from end thereof forming said first hole; said wind collecting hood device comprises a conical curved tube curved and tapered from a lower side to an upper side of said wind collecting hood device, said conical curved tube of said wind collecting hood device extending to the smallest tube diameter and the end of the smallest tube diameter forming said second hole, said conical curved tube being combined inside said cavity.

11. The hand-held operation cleaning, sterilization and disinfection atomizing diffuser as claimed in claim 9, wherein said cavity, first hole, said wind collecting hood device and said second hole are pitched upward at an angle relative to said housing.

12. The hand-held operation cleaning, sterilization and disinfection atomizing diffuser as claimed in claim 10, wherein said cavity, first hole, said wind collecting hood device and said second hole are pitched upward at an angle relative to said housing.

13. The hand-held operation cleaning, sterilization and disinfection atomizing diffuser as claimed in claim 1, wherein said first liquid delivery pipe comprises a gravity ball with a hole and a fixing plug, said first liquid delivery pipe having one end thereof combined with said gravity ball, said first liquid delivery pipe being connected to an inner side of said water outlet, said gravity ball movably maintaining the said one end of said first liquid delivery pipe on a bottom side of said liquid storage tank by gravity.

14. The hand-held operation cleaning, sterilization and disinfection atomizing diffuser as claimed in claim 1, further comprising a light-transmissive mid-transparent umbrella-shaped cover and a mid-pass diffuser, wherein said control unit comprises a light source; said control unit, said light-transmissive mid-transparent umbrella-shaped cover and said mid-pass diffuser are combined inside said first hole; said mid-pass diffuser is combined with said light source of said control unit; said light-transmissive mid-transparent umbrella-shaped cover is combined with an upper side of said mid-pass diffuser.
